(19) United States
(12) Patent Application Publication
(10) Pub. No.: US 2017/0225003 A1
(43) Pub. Date: Aug. 10, 2017

(54) APPARATUS AND METHOD FOR TARGETED DRUG DELIVERY

(71) Applicant: KONINKLIJKE PHILIPS N.V., EINDHOVEN (NL)

(72) Inventor: BERNHARD GLEICH, EINDHOVEN (NL)

(73) Assignee: Koninklijke Philips N.V.

(21) Appl. No.: 15/502,295

(22) PCT Filed: Jul. 28, 2015
(86) PCT No.: PCT/EP2015/067197

§ 371 Date: Feb. 7, 2017

(30) Foreign Application Priority Data
Aug. 13, 2014 (EP) 14180753.7

(51) Int. Cl. A61N 1/40; A61N 2/00; A61N 2/02; A61K 41/00

(52) U.S. Cl. CPC A61N 1/406; A61K 41/0052; A61N 2/002; A61N 2/004; A61N 2/02; A61K 47/4893

(57) ABSTRACT

The present invention relates to an apparatus and a method for targeted drug delivery by use of a drug substance comprising a drug and magnetic particles. The apparatus comprises selection means for generating a magnetic selection field (50) having a pattern in space of its magnetic field strength such that a first sub-zone (52) having a low magnetic field strength where the magnetization of the magnetic particles is not saturated and a second sub-zone having a higher magnetic field strength where the magnetization of the magnetic particles is saturated are formed in a field of view (28), drive means for changing the position in space of the two sub-zones (52, 54) in the field of view (28) by means of a magnetic drive field so that the magnetization of the magnetic particles changes locally, and a control unit (150) for controlling said drive means to change the position in space of the two sub-zones (52, 54) such that after administration of the drug substance the first sub-zone (52) is moved through a surrounding area (320) arranged around a target area (310) except through the target area (310) itself, said surrounding area (320) representing a potentially affected volume and/or having a predetermined maximal distance from said target area (310).

### FIELD OF THE INVENTION

The present invention relates to an apparatus and a method for targeted drug delivery by use of a drug substance comprising a drug and magnetic particles. Further, the present invention relates to a computer program for implementing said method on a computer and for controlling such an apparatus.

### BACKGROUND OF THE INVENTION

Local drug delivery by magnetic means has been already demonstrated using strong magnetic gradient fields, as described in “Locoregional cancer treatment with magnetic drug targeting” by Alexiou C, Arnold W, Klein R J, Parak F G, Hulin P, Bergemann C, Erhardt W, Wagenpfeil S, Lübbe A S, Institute of Physics Publishing Journal of Physics: Condensed Matter 18 (2006) S2893-S2902. The strong magnetic gradients trap the particles in the vascular system and enable a higher fraction to leave the vessel eventually. The particles may be coupled to therapeutic agents that are released at the targeted tissue site, where they are retained by magnetic fields long enough time for the agents to act on the tissue. The main drawback of this approach is that magnetic gradient fields are strongest near the surface of the patient so that targeting of a confined region is not possible.

### SUMMARY OF THE INVENTION

It is an object of the present invention to provide an apparatus and a method for targeted drug delivery by use of a drug substance comprising a drug and magnetic particles which enable to target the drug delivery more precisely to a confined region.

In a first aspect of the present invention an apparatus configured for targeted drug delivery by use of a drug substance comprising a drug and magnetic particles is presented, which apparatus comprises:

selection means comprising a selection field signal generator unit and selection field elements for generating a magnetic selection field having a pattern in space of its magnetic field strength such that a first sub-zone having a low magnetic field strength where the magnetization of the magnetic particles is not saturated and a second sub-zone having a higher magnetic field strength where the magnetization of the magnetic particles is saturated are formed in a field of view,

drive means comprising a drive field signal generator unit and a drive coil, said drive coil being configured for changing the position in space of the two sub-zones in the field of view by means of a magnetic drive field so that the magnetization of the magnetic particles changes locally,

a control unit for controlling said drive means to change the position in space of the two sub-zones such that after administration of the drug substance the first sub-zone is moved through a surrounding area arranged around a target area except through the target area itself, said surrounding area representing a potentially affected volume and/or having a predetermined maximal distance from said target area.

In a further aspect of the present invention a corresponding method for controlling an apparatus for targeted drug delivery by use of a drug substance comprising a drug and magnetic particles is presented.

In yet a further aspect of the present invention a computer program is presented comprising program code means for causing a computer to control an apparatus as according to the present invention to carry out the steps of the method proposed according to the present invention when said computer program is carried out on the computer.

Preferred embodiments of the invention are defined in the dependent claims. It shall be understood that the claimed method and the claimed computer program have similar and/or identical preferred embodiments as the claimed apparatus and as defined in the dependent claims.

The present invention is based on the idea to make use of a time varying magnetic gradient field that allows, at least partly, the release of the magnetic particles in all locations except the target area, at which e.g. a tumor is located. This varying magnetic gradient field has a substantially field free point (FFP) or field free line (FFL), defined as the point or line where the magnetic field is minimum, within a zone (called the “first sub-zone”) delimiting a volume in which the magnetic fields have values sufficiently low such that the magnetization of the magnetic particles in this zone are not saturated and that the magnetic particles in this zone cannot be magnetically hold against the resulting other forces acting on those particles (e.g. blood flow stream in blood vessels). In the following, reference is generally made to the FFP, which will be extended to the said “first sub-zone”, and it shall be understood that a FFL can generally be used instead of a FFP, even if only an FFP is explicitly mentioned, as well as a first sub-zone associated with such a FFL.

A force imposed on the magnetic particles depends on the distance to the FFP (or FFL). This can be reached by using suitable magnetic particles (e.g. multi-domain particles and/or a cluster of smaller magnetic particles) having a magnetic field dependent magnetization, such as particles made for example from paramagnetic material, and by superimposing a homogeneous, very high frequency magnetic field (referred to as “magnetic drive field”, e.g. moved along a trajectory in a 3D Lissajous pattern to average forces). If the FFP is near a specific location, the magnetic particles (and so the drug) at this location are released, i.e. no holding force is applied any longer on these magnetic particles so that they can be moved away from this specific location by the blood stream within the blood vessels in this area.

After administration of a bolus of the drug substance including the magnetic particles the FFP is scanned over all tissue (referred to as “surrounding area” surrounding the “target area”), where the drug substance could reach a harmful concentration, but excluding the tissue (i.e. the target area), where the harmful concentration is desired. In order to have a low concentration of the drug substance in the surrounding area, the FFP is moved through the surrounding area sufficiently slowly and along a sufficiently dense path, so that preferably all regions of the surrounding area are successively or simultaneously hit by a first sub-zone (where the particles are released) once or multiple times. The FFP is thus moved such that it does never move through the target area, where the concentration shall be maintained at a higher level due to the maintained holding forces in place as explained before and since the drug substance is not released there so that it can be moved away by the blood stream. In case of using an FFL, the FFL is moved such that it is at most tangential to the target are, but does never cross the target area. The magnetic particles, and thus the drug substance, are thus trapped in the target area.

This procedure may be repeated, until the concentration in the tissue in the surrounding area, that should not be harmed, stays low enough. If necessary, the magnetic gradient field is maintained, until all the payload of the drug substance is delivered or the drug is firmly attached to the tissue.

The proposed apparatus can make use of elements of a known Magnetic Particle Imaging (MPI) apparatus, wherein the control unit and thus the scanning sequence changes, as long as the gradient strength is strong enough.

MPI is an emerging medical imaging modality. The first versions of MPI were two-dimensional in that they produced two-dimensional images. Newer versions are three-dimensional (3D). A four-dimensional image of a non-static object can be created by combining a temporal sequence of 3D images to a movie, provided the object does not significantly change during the data acquisition for a single 3D image.

MPI is a reconstructive imaging method, like Computed Tomography (CT) or Magnetic Resonance Imaging (MRI). Accordingly, an MP image of an object's volume of interest is generated in two steps. The first step, referred to as data acquisition, is performed using an MPI scanner. The MPI scanner may have means to generate a static (or slowly varying) magnetic gradient field, called the “selection field”, which has a (single or more) field-free point(s) (FFP(s)) or a field-free line (FFL) at the isocenter of the scanner. Moreover, this FFP (or the FFL; mentioning “FFP” in the following shall generally be understood as meaning FFP or FFL) is surrounded by a first sub-zone with a low magnetic field strength, which is in turn surrounded by a second sub-zone with a higher magnetic field strength. In addition, the scanner has means to generate a time-dependent, spatially nearly homogeneous magnetic field. Actually, this field can obtained by superposing a rapidly changing field, called the “drive field”, with the selection field. The region of interest may be spread over a much larger surface or volume thanks to the addition of a third type of field, called the “focus field”, which is varying more slowly with a larger amplitude than the drive field. By adding the time-dependent drive (and focus) field(s) to the static selection field, the FFP may be moved along a predetermined FFP trajectory throughout a “volume of scanning” surrounding the isocenter. The scanner also has typically an arrangement of one or more, e.g. three, receive coils and can record any voltages induced in these coils. For the data acquisition, the object to be imaged is placed in the scanner such that the object's volume of interest is enclosed by the scanner's field of view, which is a subset of the volume of scanning

The object must contain magnetic nanoparticles or other magnetic non-linear materials; if the object is an animal or a patient, a tracer containing such particles is administered to the animal or patient prior to the scan. During the data acquisition, the MPI scanner moves the FFP along a deliberately chosen trajectory that intends to trace out/cover the volume of scanning, or at least the field of view. The magnetic nanoparticles within the object experience a changing magnetic field and respond by changing their magnetization. The changing magnetization of the nanoparticles induces a time-dependent voltage (or another type of signal or parameters) in each of the receive coils. This voltage is sampled in a receiver associated with the receive coil. The samples output by the receivers are recorded and constitute the acquired data. The parameters that control the details of the data acquisition make up the “scan protocol”.

Such an MPI apparatus and method have the advantage that they can be used to examine arbitrary examination objects—e. g. human bodies—in a non-destructive manner and with a high spatial resolution, both close to the surface and remote from the surface of the examination object. Such an apparatus and method are generally known and have been first described in DE 101 51 778 A1 and in Gleich, B. and Weizenecker, J. (2005), “Tomographic imaging using the nonlinear response of magnetic particles” in Nature, vol. 435, pp. 1214-1217, in which also the reconstruction principle is generally described. The apparatus and method for magnetic particle imaging (MPI) described in that publication take advantage of the non-linear magnetization curve of small magnetic particles. Meanwhile a number of patent documents have been published disclosing the general technology used by MPI. Applications of MPI and corresponding apparatuses and methods are also described in WO 2011/030276 A1, WO 2013/080145 A1 and WO 2012/046157 A1.

In an embodiment the proposed apparatus can be used for image reconstruction. For this purpose the apparatus preferably further comprises a receiving means comprising a signal receiving unit and a receiving coil, said receiving coil being configured for acquiring detection signals, which detection signals depend on the magnetization in the field of view, which magnetization is influenced by the change in the position in space of the first and second sub-zone. In an advantageous space-saving embodiment said drive means and said receiving means are combined into drive and receiving means comprising a drive-receiving coil, said drive-receiving coil being configured both for changing the position in space of the two sub-zones in the field of view by means of a magnetic drive field and for acquiring detection signals.

In the context of the present invention, the apparatus preferably further comprises a processing unit for reconstructing the spatial distribution and/or concentration of the magnetic particles within the surrounding area from the detection signals and for determining if the concentration of the drug substance within the surrounding area is below a predetermined threshold, in which case the gradient of the magnetic gradient field can be reduced or completely removed.

Generally, for image reconstruction by use of the proposed magnetic particle imaging apparatus and method the magnetic gradient field (i.e. the magnetic selection field) is generated with a spatial distribution of the magnetic field strength such that the field of view comprises a first sub-area with lower magnetic field strength (e.g. the FFP), the lower magnetic field strength being adapted such that the magnetization of the magnetic particles located in the first sub-area is not saturated, and a second sub-area with a higher magnetic field strength, the higher magnetic field strength being adapted such that the magnetization of the magnetic particles located in the second sub-area is saturated. Due to the non-linearity of the magnetization characteristic curve of the magnetic particles the magnetization and thereby the magnetic field generated by the magnetic particles shows higher harmonics, which, for example, can be detected by a detection coil. The evaluated signals (the higher harmonics of the signals) contain information about the spatial distribution of the magnetic particles, which again can be used e.g. for medical imaging, for the visualization of the spatial distribution of the magnetic particles and/or for other applications.

Thus, the apparatus and the method according to the present invention are based on a new physical principle (i.e. the principle referred to as MPI) that is different from other known conventional medical imaging techniques, as for example nuclear magnetic resonance (NMR). In particular, this new MPI-principle, does, in contrast to NMR, not exploit the influence of the material on the magnetic resonance characteristics of protons, but rather directly detects the magnetization of the magnetic material by exploiting the non-linearity of the magnetization characteristic curve. In particular, the MPI-technique exploits the higher harmonics of the generated magnetic signals which result from the non-linearity of the magnetization characteristic curve in the area where the magnetization changes from the non-saturated to the saturated state.

It shall, however, be noted that for the main application of the present invention, i.e. for targeted drug delivery, the changes of the magnetic drive field need not be as fast as conventionally used in an MPI apparatus, where the magnetic drive field changes so fast that the generated harmonics can be received and used for image reconstruction.

According to a preferred embodiment said control unit is configured to control said drive means to change the position in space of the two sub-zones such that the first sub-zone is moved one or multiple times around said target area. This ensures that drug substance is removed or released from the surrounding area around the target area.

Preferably, said control unit is configured to control said drive means to change the position in space of the two sub-zones such that the first sub-zone scans the surrounding area around the target area, whereby the direction of movement and/or the path of the first sub-zone is changed several times. This further improves the removal or release of the drug substance from the surrounding area.

In an embodiment said control unit is configured to control said drive means and/or said selection means (the latter is to provide a stationary magnetic field, in particular a stationary magnetic gradient field, over the target area and the surrounding area during administration of the drug substance). In this way, the drug substance, which is preferably administered close to or even into the target area, but at least into the surrounding area, gets trapped within the target area and the surrounding area during the administration of the drug substance. Afterwards, when the first sub-zone is moved through the surrounding area, the stationary magnetic field will be removed or decreased or released from the surrounding area, but preferably will remain within the target area as desired, which supports that the drug substance remains at a sufficiently high concentration within the target area and is not washed away by the blood stream with the blood vessels within this target area.

To provide a stationary magnetic gradient field the selection means and/or the drive means may be used. In one embodiment the position in space of the two sub-zones is provided such that the first sub-zone is located outside the surrounding area and outside the target area, e.g. at the surface of the subject's body, during administration of the drug substance. Hence, a magnetic gradient field is achieved across the subject's body.

Preferably, said control unit is configured to control said drive means to move the first sub-zone through the surrounding area immediately after the administration of the drug substance. Otherwise, the drug within the drug substance may start developing its drug effect which is generally undesired in the surrounding, in particular if the concentration of the drug substance is too high in the surrounding area. Hence, according to this embodiment the concentration of the drug substance in the surrounding area is quickly reduced.

Preferably, said control unit is configured to control said drive means to change the position in space of the two sub-zones such that the first sub-zone is moved through the surrounding area until the concentration of the drug substance within the surrounding area is below a predetermined threshold. Hence, the drug substance is preferably moved to or kept within the target area, or free to move within the surrounding area when it is in the first sub-zone and typically in the direction of the blood stream within blood vessels (since the drug substance is magnetically released when the FFP moves over it). This embodiment avoids any damages of healthy tissue within the surrounding area.

In another embodiment said selection means is configured to change the stationary gradient (e.g. its strength and/or direction) of the magnetic selection field and/or the size of the first sub-zone while the first sub-zone is moved through the surrounding area. This enables a more precise control of the removal or release of the drug substance from the surrounding area.

The present invention may generally be used for targeted delivery of any kinds of drug, but preferably said drug substance comprises an anticancer agents as drug, in particular radionuclides, cancer-specific antibodies, cytostatika and/or genes.

Preferably, said drug substance comprises multi-domain magnetic particles or a cluster of particles to be able to provide a sufficient force on the drug substance for moving it into a desired direction, since the force depends on the distance of the magnetic particles from the first sub-zone (i.e. the FFP). Further, for this purpose said multi-domain magnetic particles or cluster of particles preferably have a volume of substantially a sphere having a diameter of at least 100 nm, in particular of at least 1 μm.

### BRIEF DESCRIPTION OF THE DRAWINGS

These and other aspects of the invention will be apparent from and elucidated with reference to the embodiment(s) described hereinafter. In the following drawings

FIG. 1 shows a first embodiment of an MPI apparatus,

FIG. 2 shows an example of the selection field pattern produced by an apparatus as shown in FIG. 1,

FIG. 3 shows a second embodiment of an MPI apparatus,

FIG. 4 shows a third and a fourth embodiment of an MPI apparatus,

FIG. 5 shows a block diagram of an MPI apparatus according to the present invention, and

FIG. 6 shows diagram illustrating targeted drug delivery according to the present invention.

### DETAILED DESCRIPTION OF THE INVENTION

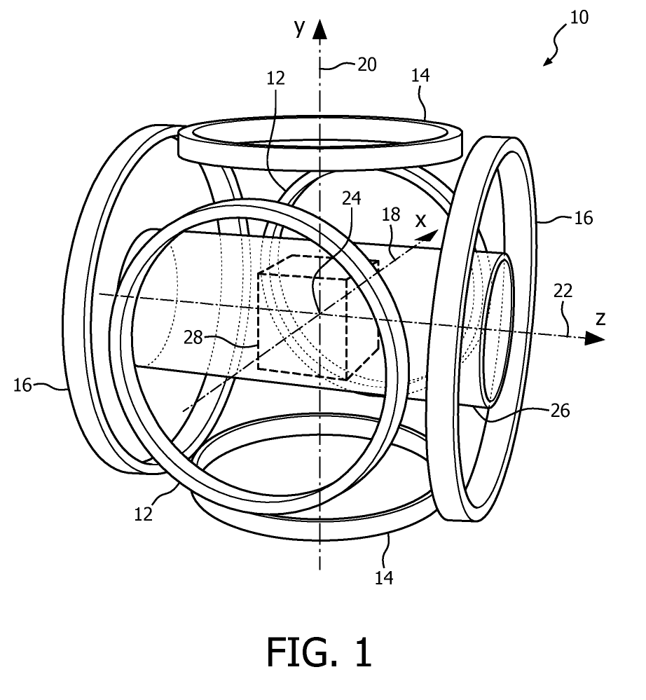

Before the details of the present invention shall be explained, basics of magnetic particle imaging shall be explained in detail with reference to FIGS. 1 to 4. In particular, several embodiments of an MPI scanner for medical diagnostics will be described. An informal description of the data acquisition will also be given. The similarities and differences between the different embodiments will be pointed out. Generally, the present invention can be used in all these different embodiments of an MPI apparatus, i.e. the hardware of such an MPI apparatus can be used for the proposed targeted drug delivery of a drug substance to a target area, such as a tumor.

The first embodiment 10 of an MPI scanner shown in FIG. 1 has three pairs 12, 14, 16 of coaxial parallel circular coils, these coil pairs being arranged as illustrated in FIG. 1. These coil pairs 12, 14, 16 serve to generate the selection field as well as the drive and focus fields. The axes 18, 20, 22 of the three coil pairs 12, 14, 16 are mutually orthogonal and meet in a single point, designated the isocenter 24 of the MPI scanner 10. In addition, these axes 18, 20, 22 serve as the axes of a 3D Cartesian x-y-z coordinate system attached to the isocenter 24. The vertical axis 20 is nominated the y-axis, so that the x- and z-axes are horizontal. The coil pairs 12, 14, 16 are named after their axes. For example, the y-coil pair 14 is formed by the coils at the top and the bottom of the scanner. Moreover, the coil with the positive (negative) y-coordinate is called the y⁺-coil (y⁻-coil), and similarly for the remaining coils. When more convenient, the coordinate axes and the coils shall be labelled with x₁, x₂, and x₃, rather than with x, y, and z.

The scanner 10 can be set to direct a predetermined, time-dependent electric current through each of these coils 12, 14, 16, and in either direction. If the current flows clockwise around a coil when seen along this coil's axis, it will be taken as positive, otherwise as negative. To generate the static selection field, a constant positive current I^{S} is made to flow through the z⁺-coil, and the current −I^{S} is made to flow through the z⁻-coil. The z-coil pair 16 then acts as an anti-parallel circular coil pair.

It should be noted here that the arrangement of the axes and the nomenclature given to the axes in this embodiment is just an example and might also be different in other embodiments. For instance, in practical embodiments the vertical axis is often considered as the z-axis rather than the y-axis as in the present embodiment. This, however, does not generally change the function and operation of the device and the effect of the present invention.

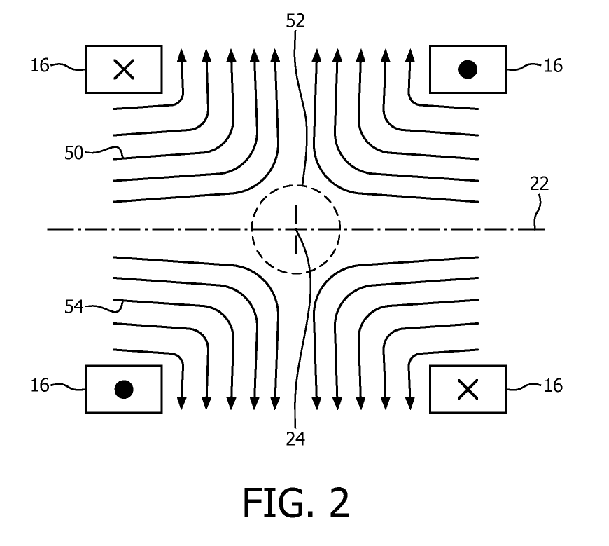

The magnetic selection field, which is generally a magnetic gradient field, is represented in FIG. 2 by the field lines 50. It has a substantially constant gradient in the direction of the (e.g. horizontal) z-axis 22 of the z-coil pair 16 generating the selection field and reaches the value zero in the isocenter 24 on this axis 22. Starting from this field-free point (not individually shown in FIG. 2), the field strength of the magnetic selection field 50 increases in all three spatial directions as the distance increases from the field-free point. In a first sub-zone or region 52 which is denoted by a dashed line around the isocenter 24 the field strength is so small that the magnetization of particles present in that first sub-zone 52 is not saturated, whereas the magnetization of particles present in a second sub-zone 54 (outside the region 52) is in a state of saturation. In the second sub-zone 54 (i.e. in the residual part of the scanner's field of view 28 outside of the first sub-zone 52) the magnetic field strength of the selection field is sufficiently strong to keep the magnetic particles in a state of saturation.

By changing the position of the two sub-zones 52, 54 (including the field-free point) within the field of view 28 the (overall) magnetization in the field of view 28 changes. By determining the magnetization in the field of view 28 or physical parameters influenced by the magnetization, information about the spatial distribution of the magnetic particles in the field of view 28 can be obtained. In order to change the relative spatial position of the two sub-zones 52, 54 (including the field-free point) in the field of view 28, further magnetic fields, i.e. the magnetic drive field, and, if applicable, the magnetic focus field, are superposed to the selection field 50.

To generate the drive field, a time dependent current I^{D}₁ is made to flow through both x-coils 12, a time dependent current I^{D}₂ through both y-coils 14, and a time dependent current I^{D}₃ through both z-coils 16. Thus, each of the three coil pairs acts as a parallel circular coil pair. Similarly, to generate the focus field, a time dependent current I^{F}₁ is made to flow through both x-coils 12, a current I^{F}₂ through both y-coils 14, and a current I^{F}₃ through both z-coils 16.

It should be noted that the z-coil pair 16 is special: It generates not only its share of the drive and focus fields, but also the selection field (of course, in other embodiments, separate coils may be provided). The current flowing through the z^{±}-coil is I^{D}₃+I^{F}₃±I^{S}. The current flowing through the remaining two coil pairs 12, 14 is I^{d}ₖ+I^{F}ₖ, k=1, 2. Because of their geometry and symmetry, the three coil pairs 12, 14, 16 are well decoupled. This is wanted.

Being generated by an anti-parallel circular coil pair, the selection field is rotationally symmetric about the z-axis, and its z-component is nearly linear in z and independent of x and y in a sizeable volume around the isocenter 24. In particular, the selection field has a single field-free point (FFP) at the isocenter. In contrast, the contributions to the drive and focus fields, which are generated by parallel circular coil pairs, are spatially nearly homogeneous in a sizeable volume around the isocenter 24 and parallel to the axis of the respective coil pair. The drive and focus fields jointly generated by all three parallel circular coil pairs are spatially nearly homogeneous and can be given any direction and strength, up to some maximum strength. The drive and focus fields are also time-dependent. The difference between the focus field and the drive field is that the focus field varies slowly in time and may have a large amplitude, while the drive field varies rapidly and has a small amplitude. There are physical and biomedical reasons to treat these fields differently. A rapidly varying field with a large amplitude would be difficult to generate and potentially hazardous to a patient.

The embodiment 10 of the MPI scanner has at least one further pair, preferably three further pairs, of parallel circular coils, again oriented along the x-, y-, and z-axes. These coil pairs, which are not shown in FIG. 1, serve as receive coils. As with the coil pairs 12, 14, 16 for the drive and focus fields, the magnetic field generated by a constant current flowing through one of these receive coil pairs is spatially nearly homogeneous within the field of view and parallel to the axis of the respective coil pair. The receive coils are supposed to be well decoupled. The time-dependent voltage induced in a receive coil is amplified and sampled by a receiver attached to this coil. More precisely, to cope with the enormous dynamic range of this signal, the receiver samples the difference between the received signal and a reference signal. The transfer function of the receiver is non-zero from zero Hertz (“DC”) up to the frequency where the expected signal level drops below the noise level. Alternatively, the MPI scanner has no dedicated receive coils. Instead the drive field transmit coils are used as receive coils as is the case according to the present invention using combined drive-receiving coils.

The embodiment 10 of the MPI scanner shown in FIG. 1 has a cylindrical bore 26 along the z-axis 22, i.e. along the axis of the selection field. All coils are placed outside this bore 26. For the data acquisition, the patient (or object) to be imaged is placed in the bore 26 such that the patient's volume of interest—that volume of the patient (or object) that shall be imaged—is enclosed by the scanner's field of view 28—that volume of the scanner whose contents the scanner can image. The patient (or object) is, for instance, placed on a patient table. The field of view 28 is a geometrically simple, isocentric volume in the interior of the bore 26, such as a cube, a ball, a cylinder or an arbitrary shape. A cubical field of view 28 is illustrated in FIG. 1.

The patient's volume of interest is supposed to contain magnetic nanoparticles. Prior to the diagnostic imaging of and/or targeted drug delivery to, for example, a tumor, the magnetic particles are brought to the volume of interest, e.g. by means of a liquid comprising the magnetic particles which is injected into the body of the patient (object) or otherwise administered, e.g. orally, to the patient.

Generally, various ways for bringing the magnetic particles into the field of view exist. In particular, in case of a patient into whose body the magnetic particles are to be introduced, the magnetic particles can be administered by use of surgical and non-surgical methods, and there are both methods which require an expert (like a medical practitioner) and methods which do not require an expert, e.g. can be carried out by laypersons or persons of ordinary skill or the patient himself/herself. Among the surgical methods there are potentially non-risky and/or safe routine interventions, e.g. involving an invasive step like an injection of a tracer into a blood vessel (if such an injection is at all to be considered as a surgical method), i.e. interventions which do not require considerable professional medical expertise to be carried out and which do not involve serious health risks. Further, non-surgical methods like swallowing or inhalation can be applied.

Generally, the magnetic particles are pre-delivered or pre-administered before the actual steps of data acquisition are carried out. In embodiments, it is, however, also possible that further magnetic particles are delivered/administered into the field of view.

An embodiment of magnetic particles comprises, for example, a spherical substrate, for example, of glass which is provided with a soft-magnetic layer which has a thickness of, for example, 5 nm and consists, for example, of an iron-nickel alloy (for example, Permalloy). This layer may be covered, for example, by means of a coating layer which protects the particle against chemically and/or physically aggressive environments, e.g. acids. The magnetic field strength of the magnetic selection field 50 required for the saturation of the magnetization of such particles is dependent on various parameters, e.g. the diameter of the particles, the used magnetic material for the magnetic layer and other parameters.

In practice, magnetic particles commercially available under the trade name Resovist (or similar magnetic particles) are often used, which have a core of magnetic material or are formed as a massive sphere and which have a diameter in the range of nanometers, e.g. 40 or 60 nm.

For further details of the generally usable magnetic particles and particle compositions, the corresponding parts of EP 1224542, WO 2004/091386, WO 2004/091390, WO 2004/091394, WO 2004/091395, WO 2004/091396, WO 2004/091397, WO 2004/091398, WO 2004/091408 are herewith referred to, which are herein incorporated by reference. In these documents and other patent applications of the applicant more details of the MPI method in general can be found as well.

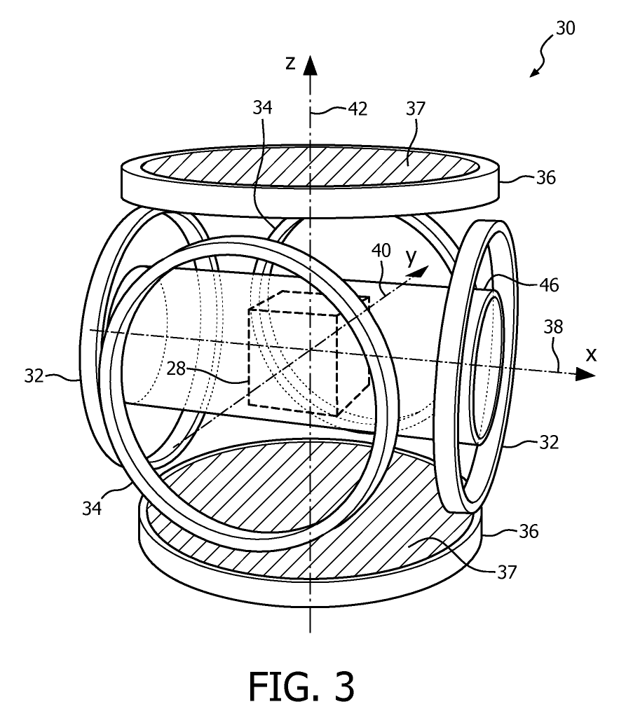

Like the first embodiment 10 shown in FIG. 1, the second embodiment 30 of the MPI scanner shown in FIG. 3 has three circular and mutually orthogonal coil pairs 32, 34, 36, but these coil pairs 32, 34, 36 generate the selection field and the focus field only. The z-coils 36, which again generate the selection field, are filled with ferromagnetic material 37. The z-axis 42 of this embodiment 30 is oriented vertically, while the x- and y-axes 38, 40 are oriented horizontally. The bore 46 of the scanner is parallel to the x-axis 38 and, thus, perpendicular to the axis 42 of the selection field. The drive field is generated by a solenoid (not shown) along the x-axis 38 and by pairs of saddle coils (not shown) along the two remaining axes 40, 42. These coils are wound around a tube which forms the bore. The drive field coils also serve as receive coils.

To give a few typical parameters of such an embodiment: The z-gradient of the selection field, G, has a strength of G/μ₀=2.5 T/m, where μ₀ is the vacuum permeability. The temporal frequency spectrum of the drive field is concentrated in a narrow band around 25 kHz (up to approximately 250 kHz). The useful frequency spectrum of the received signals lies between 50 kHz and 1 MHz (eventually up to approximately 15 MHz). The bore has a diameter of 120 mm. The biggest cube 28 that fits into the bore 46 has an edge length of 120 mm/√2≈84 mm.

Since the construction of field generating coils is generally known in the art, e.g. from the field of magnetic resonance imaging, this subject need not be further elaborated herein.

In an alternative embodiment for the generation of the selection field, permanent magnets (not shown) can be used. In the space between two poles of such (opposing) permanent magnets (not shown) there is formed a magnetic field which is similar to that shown in FIG. 2, that is, when the opposing poles have the same polarity. In another alternative embodiment, the selection field can be generated by a mixture of at least one permanent magnet and at least one coil.

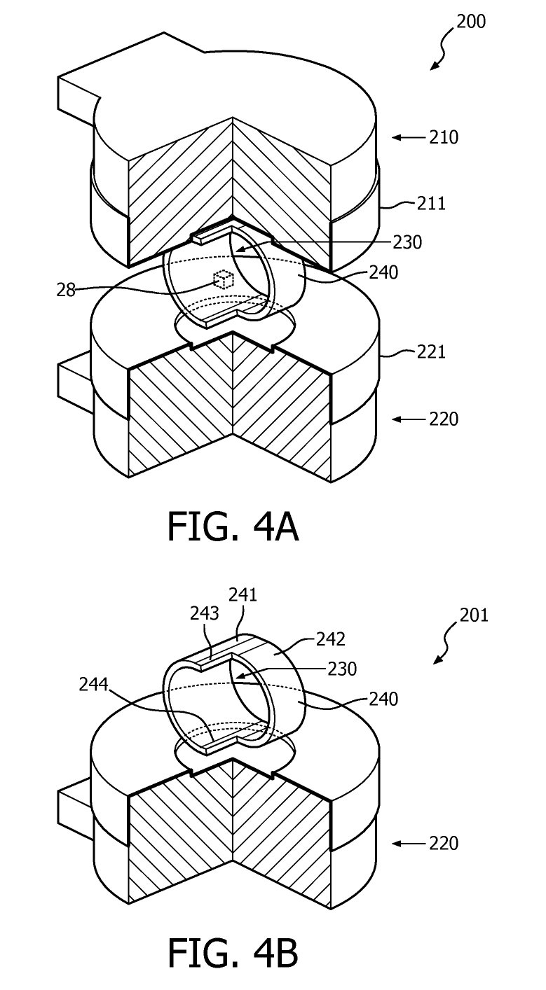

FIG. 4 shows two embodiments of the general outer layout of an MPI apparatus 200, 300. FIG. 4A shows an embodiment of the proposed MPI apparatus 200 comprising two selection-and-focus field coil units 210, 220 which are basically identical and arranged on opposite sides of the examination area 230 formed between them. Further, a drive field coil unit 240 is arranged between the selection-and-focus field coil units 210, 220, which are placed around the area of interest of the patient (not shown). The selection-and-focus field coil units 210, 220 comprise several selection-and-focus field coils for generating a combined magnetic field representing the above-explained magnetic selection field and magnetic focus field. In particular, each selection-and-focus field coil unit 210, 220 comprises a, preferably identical, set of selection-and-focus field coils. Details of said selection-and-focus field coils will be explained below.

The drive field coil unit 240 comprises a number of drive field coils for generating a magnetic drive field. These drive field coils may comprise several pairs of drive field coils, in particular one pair of drive field coils for generating a magnetic field in each of the three directions in space. In an embodiment the drive field coil unit 240 comprises two pairs of saddle coils for two different directions in space and one solenoid coil for generating a magnetic field in the longitudinal axis of the patient.

The selection-and-focus field coil units 210, 220 are generally mounted to a holding unit (not shown) or the wall of room. Preferably, in case the selection-and-focus field coil units 210, 220 comprise pole shoes for carrying the respective coils, the holding unit does not only mechanically hold the selection-and-focus field coil unit 210, 220 but also provides a path for the magnetic flux that connects the pole shoes of the two selection-and-focus field coil units 210, 220.

As shown in FIG. 4a, the two selection-and-focus field coil units 210, 220 each include a shielding layer 211, 221 for shielding the selection-and-focus field coils from magnetic fields generated by the drive field coils of the drive field coil unit 240.

In the embodiment of the MPI apparatus 201 shown in FIG. 4B only a single selection-and-focus field coil unit 220 is provided as well as the drive field coil unit 240. Generally, a single selection-and-focus field coil unit is sufficient for generating the required combined magnetic selection and focus field. Said single selection-and-focus field coil unit 220 may thus be integrated into a (not shown) patient table on which a patient is placed for the examination. Preferably, the drive field coils of the drive field coil unit 240 may be arranged around the patient's body already in advance, e.g. as flexible coil elements. In another implementation, the drive field coil unit 240 can be opened, e.g. separable into two subunits 241, 242 as indicated by the separation lines 243, 244 shown in FIG. 4b in axial direction, so that the patient can be placed in between and the drive field coil subunits 241, 242 can then be coupled together.

In still further embodiments of the MPI apparatus, even more selection-and-focus field coil units may be provided which are preferably arranged according to a uniform distribution around the examination area 230. However, the more selection-and-focus field coil units are used, the more will the accessibility of the examination area for placing a patient therein and for accessing the patient itself during an examination by medical assistance or doctors be limited.

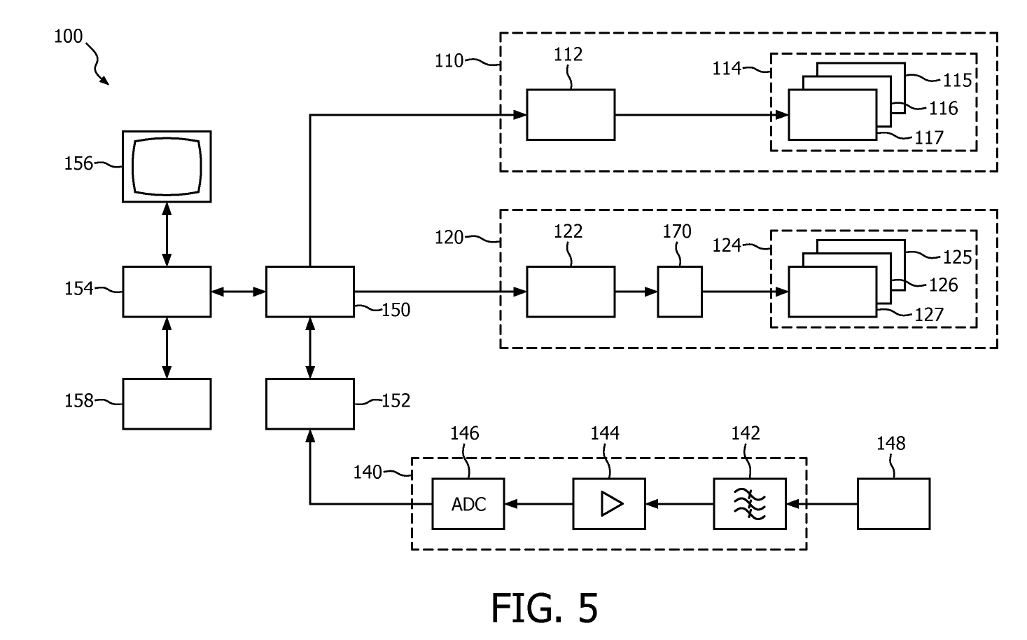

FIG. 5 shows a general block diagram of a preferred embodiment of an apparatus 100 according to the present invention. The general principles of magnetic particle imaging explained above are valid and applicable to this embodiment as well, unless otherwise specified.

The embodiment of the apparatus 100 shown in FIG. 5 comprises various coils for generating the desired magnetic fields. First, the coils and their functions in MPI shall be explained.

For generating the combined magnetic selection-and-focus field, selection-and-focus means 110 are provided. The magnetic selection-and-focus field has a pattern in space of its magnetic field strength such that the first sub-zone (52 in FIG. 2) having a low magnetic field strength where the magnetization of the magnetic particles is not saturated and a second sub-zone (54 in FIG. 4) having a higher magnetic field strength where the magnetization of the magnetic particles is saturated are formed in the field of view 28, which is a small part of the examination area 230, which is conventionally achieved by use of the magnetic selection field. Further, by use of the magnetic selection-and-focus field the position in space of the field of view 28 within the examination area 230 can be changed, as conventionally done by use of the magnetic focus field.

The selection-and-focus means 110 comprises at least one set of selection-and-focus field coils 114 and a selection-and-focus field generator unit 112 for generating selection-and-focus field currents to be provided to said at least one set of selection-and-focus field coils 114 (representing one of the selection-and-focus field coil units 210, 220 shown in FIGS. 4A, 4B) for controlling the generation of said magnetic selection-and-focus field. Preferably, a separate generator subunit is provided for each coil element (or each pair of coil elements) of the at least one set of selection-and-focus field coils 114. Said selection-and-focus field generator unit 112 comprises a controllable current source (generally including an amplifier) and a filter unit which provide the respective coil element with the field current to individually set the gradient strength and field strength of the contribution of each coil to the magnetic selection-and-focus field. It shall be noted that the filter unit 114 can also be omitted. Further, separate focus and selection means are provided in other embodiments.

For generating the magnetic drive field the apparatus 100 further comprises drive means 120 comprising a drive field signal generator unit 122 and a set of drive field coils 124 (representing the drive coil unit 240 shown in FIGS. 4A, 4B) for changing the position in space and/or size of the two sub-zones in the field of view by means of a magnetic drive field so that the magnetization of the magnetic material changes locally. As mentioned above said drive field coils 124 preferably comprise two pairs 125, 126 of oppositely arranged saddle coils and one solenoid coil 127. Other implementations, e.g. three pairs of coil elements, are also possible.

The drive field signal generator unit 122 preferably comprises a separate drive field signal generation subunit for each coil element (or at least each pair of coil elements) of said set of drive field coils 124. Said drive field signal generator unit 122 preferably comprises a drive field current source (preferably including a power amplifier) and a filter unit for providing a time-dependent drive field current to the respective drive field coil.

As mentioned above, for the application of the apparatus 100 for targeted drug delivery, the temporal frequency spectrum of the drive field may be concentrated in a lower frequency band than conventionally used for obtaining detection signals to reconstruct an image. For example, for targeted drug deliver the frequency may be in the range around 10 Hz (up to approximately 500 kHz). In other embodiments, focus field coils, which are optionally provided in MPI apparatus for slowly moving the field of view may be used in an apparatus and method according to the present invention for providing the movement of the FFP.

The selection-and-focus field signal generator unit 112 and the drive field signal generator unit 122 are preferably controlled by a control unit 150, which preferably controls the selection-and-focus field signal generator unit 112 such that the sum of the field strengths and the sum of the gradient strengths of all spatial points of the selection field is set at a predefined level. For this purpose the control unit 150 can also be provided with control instructions by a user according to the desired application of the MPI apparatus, which, however, is preferably omitted according to the present invention.

For using the MPI apparatus 100 for determining the spatial distribution of the magnetic particles in the examination area (or a region of interest in the examination area), particularly to obtain images of said region of interest, signal detection receiving means 148, in particular a receiving coil, and a signal receiving unit 140, which receives signals detected by said receiving means 148, are provided. Preferably, three receiving coils 148 and three receiving units 140—one per receiving coil—are provided in practice, but more than three receiving coils and receiving units can be also used, in which case the acquired detection signals are not 3-dimensional but K-dimensional, with K being the number of receiving coils.

In another embodiment (not shown) the, one to three of said drive field coils 124 (or drive field coil pairs) act (simultaneously or alternately) as receiving coils for receiving detection signals so that the receiving coils 148 can be omitted. Accordingly, these drive field coils are called “drive-receiving coils” in such an embodiment.

Said signal receiving unit 140 comprises a filter unit 142 for filtering the received detection signals. The aim of this filtering is to separate measured values, which are caused by the magnetization in the examination area which is influenced by the change in position of the two part-regions (52, 54), from other, interfering signals. To this end, the filter unit 142 may be designed for example such that signals which have temporal frequencies that are smaller than the temporal frequencies with which the receiving coil 148 is operated, or smaller than twice these temporal frequencies, do not pass the filter unit 142. The signals are then transmitted via an amplifier unit 144 to an analog/digital converter 146 (ADC).

The digitalized signals produced by the analog/digital converter 146 are fed to an image processing unit (also called reconstruction means) 152, which reconstructs the spatial distribution of the magnetic particles from these signals and the respective position which the first part-region 52 of the first magnetic field in the examination area assumed during receipt of the respective signal and which the image processing unit 152 obtains from the control unit 150. The reconstructed spatial distribution of the magnetic particles is finally transmitted via the control means 150 to a computer 154, which displays it on a monitor 156. Thus, an image can be displayed showing the distribution of magnetic particles in the field of view of the examination area.

In other applications of the MPI apparatus 100, e.g. for influencing the magnetic particles (for instance for a hyperthermia treatment) or for targeted drug delivery as proposed according to the present invention, the receiving means may also be omitted or simply not used. They may, however, be used to monitor or check the development and/or result of the drug delivery during and/or after the delivery.

Further, an input unit 158 may optionally be provided, for example a keyboard. A user may therefore be able to set the desired direction of the highest resolution and in turn receives the respective image of the region of action on the monitor 156. If the critical direction, in which the highest resolution is needed, deviates from the direction set first by the user, the user can still vary the direction manually in order to produce a further image with an improved imaging resolution. This resolution improvement process can also be operated automatically by the control unit 150 and the computer 154. The control unit 150 in this embodiment sets the gradient field in a first direction which is automatically estimated or set as start value by the user. The direction of the gradient field is then varied stepwise until the resolution of the thereby received images, which are compared by the computer 154, is maximal, respectively not improved anymore. The most critical direction can therefore be found respectively adapted automatically in order to receive the highest possible resolution.

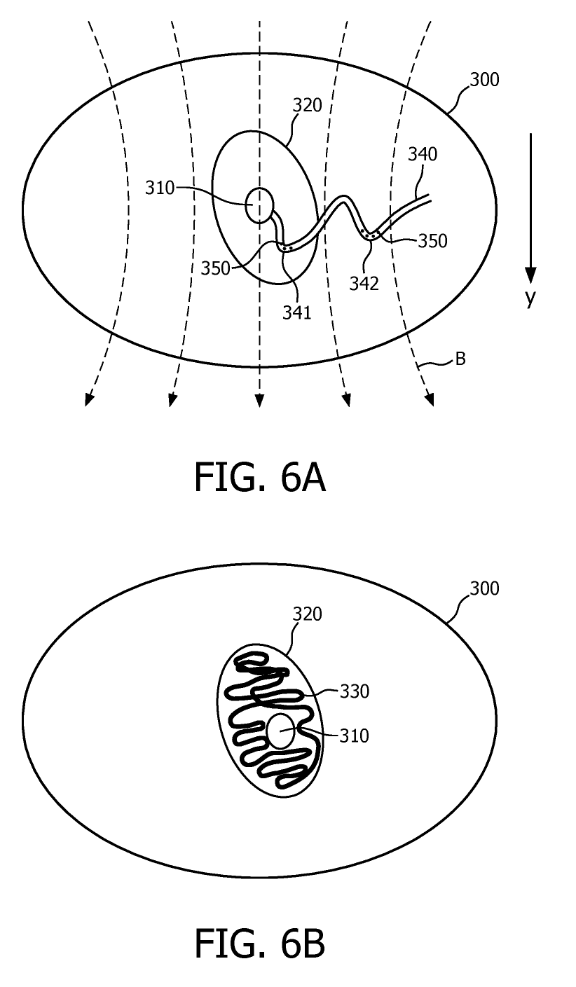

According to the present invention the control unit 150 is configured to controlling said drive means to change the position in space of the two sub-zones (52, 54) such that after administration of the drug substance the first sub-zone (52) is moved through a surrounding area arranged around a target area except through the target area itself, said surrounding area representing a potentially affected volume and/or having a predetermined maximal distance from said target area. This will be explained with reference to FIG. 6 showing a cross-sectional view of the patient's body 300 including a target area 310, in which a tumor may be located, and a surrounding area 320 surrounding said target area 310 and representing a potentially affected volume. FIG. 6A shows an initial state during administration of the drug substance and FIG. 6B shows a subsequent state after administration of the drug substance while the FFP is moved through the surrounding area 320.

An embodiment of a method for targeted drug delivery using such an apparatus would be performed as follows. The drug substance (also called agent) contains a drug and magnetic particles. Such a drug may e.g. be an anticancer agent, in particular radionuclides, cancer-specific antibodies, cytostatika and/or genes. The magnetic particles may e.g. be multi-domain magnetic particles or a cluster of particles, which have a volume of substantially a sphere having a diameter of at least 100 nm, in particular of at least 1 μm. Such types of a drug substance, which may be used according to the present invention, are e.g. disclosed in the above mentioned paper of Alexiou. In another embodiment iron/carbon particles may be used for drug delivery, using an external magnetic field to direct the drug substance to the target area inside the body. Exemplary embodiments for a drug substance are also described in U.S. Pat. No. 5,651,989 A.

In a first stage, illustrated in FIG. 6A, the drug substance is released (i.e. administered) to the arterial blood stream as close to the tumor as possible. This is done e.g. by a catheter. Hereby, “as close as possible” means that the whole tumor is fed by the blood in the target area 310, but the total blood stream is minimal or the catheter cannot go closer to the tumor. Alternatively, the drug substance may generally be administered at a different location of the body and may be moved to the target area through the vessel system by use of magnetic forces generated by magnetic fields generated by the available coils. The movement of magnetic particles or devices provided with magnetic material through the blood vessel system by use of an MPI apparatus is generally known in the art and is e.g. described in WO 2011/030276 A1.

During release of the drug substance, a stationary magnetic field B (or a stationary magnetic gradient field) is constantly switched on. This may be provided by the magnetic selection field generated by selection field coils or selection-and-focus-field coils. Alternatively, a magnetic field with a field free point may be generated with a field free point sufficiently away from the target area 310. This stationary magnetic field B provides that the magnetic particles, and thus the drug substance, are moved in one direction. If the magnetic field gradient is strong enough, the magnetic particles are trapped in the capillaries as it is likely that in the capillary system there are curves where the magnetic particles cannot leave (the magnetic particles always want to go in a direction away from the FFP or are “pulled” by the magnetic gradient, which may be comparable to a hairpin turn where the magnetic particles are trapped at the curve). This is illustrated in FIG. 6A for an exemplary capillary 340 (which is shown not to scale) having curves 341, 342 in which the drug substance 350 is trapped since the stationary magnetic field B exerts a force onto the magnetic particles in the drug substance 350 in y-direction in this example. In the capillaries, the blood sheer forces may be low enough to keep the magnetic particles in place. Hence, an intention might be to trap first all magnetic particles in the capillaries in this initial stage.

After all the drug substance is administered to the patient, a release mechanism is activated in a second stage illustrated in FIG. 6B. The forces that hold the magnetic particles in the curves 341, 342 of the capillaries are switched off or at least reduced by switching off the initial stationary magnetic field at least in the surrounding area 320 or superimposing the drive field to the stationary magnetic field. Over the target area 310 a magnetic gradient is preferably maintained to hold the drug substance in the target area 310 and avoid that the drug substance is transported away from the target area 310 by the blood stream.

While generally the magnetic field gradient at the FFP is as strong as everywhere else, the magnetic field strength is not. Hence, if the force in the magnetic particle does not depend only on the magnetic gradient, but also on the amplitude of the magnetic field, the holding forces are weakened and the magnetic particles (i.e. the drug substance) are released at a location of the FFP. This is evaluated in this second stage.

The weakening of the force is either an intrinsic property of the magnetic particles (which are e.g. multidomain particles or a cluster particles) or can be emulated by a trajectory 330 (e.g. a 3D Lissajous trajectory) with a sufficiently high frequency (up to 500 kHz of the drive field). Hence, the field free point is moved over the tissue of the surrounding area 320 that may have the drug attached to it, but which is not the tumor, i.e. the field free point is moved through the surrounding area 320 but not through the target area 310. This releases the drug substance in this area. In other words, if the FFP moves, holding force acting on the drug substances around this FFP (i.e. in the first sub-zone) would not be sufficiently high to magnetically hold the drug substances in the first sub-zone in place, and the drug substance would be therefore more free to move away from this zone, e.g. with the blood stream through the blood vessel system.

After release, the magnetic fields might be switched-off for a certain duration, such that the drug substance is free to follow the blood stream, to another location (e.g. around another target area) where the drug substance may be trapped again by applying magnetic forces according to the invention and depicted by FIGS. 6A and 6B. Alternatively, the drug substances are left without any action, following the blood stream and being naturally dissipated. Preferably, the drug substance is washed away from the surrounding area and spreads to other portions of the body through the blood vessel system, and is finally removed from the blood without leading to a high concentration anywhere within the body.

The step of moving the FFP through the surrounding area 320 is preferably repeated one or more times, optionally with different directions of movement of the FFP (e.g. along different trajectories), with different speed, with different magnetic gradients, etc. so that the drug substance is released everywhere within the surrounding area and transported away from all regions of the surrounding area as much as possible.

At the end, the drug substance may be captured by the patient's liver (or any other organ) which may then clean the blood from the drug substance. The liver toxicity of the drug needs to be sufficiently low and the volume of the target area (with the surrounding area) are preferably sufficiently small compared to the liver to act mainly only on the tumorous cells and not really on the healthy cells. As an additional option the FFP may be moved over one or more organs near the target area or the surrounding area so that the drug substance is not collected in these organs to avoid damages of them.

Preferably, the above explained acquisition and processing of detection signals by use of the conventional MPI technique may be applied in parallel or at the end to obtain the release of the drug substance is sufficient, i.e. if the amount of drug substance within the surrounding area 320 is below a predetermined threshold (set such that the tissue in the surrounding area 320 is not harmed) and/or if there are regions where too much drug substance is still present requiring further movements of the FFP over such regions.

The present invention provides a simple but effective way to precisely deliver a drug to a desired target region. Advantageously, the delivery can be monitored and/or checked afterwards with the same equipment that has been used for the delivery.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. An apparatus configured for targeted drug delivery, the apparatus comprising:
a selection apparatus, the selection apparatus comprising:
a selection field signal generator unit; and
selection field elements,
wherein the selection elements are arranged to generate a magnetic selection field,
wherein the magnetic selection field has a pattern, formed in a field of view, of its magnetic field strength such that a first sub-zone has a low magnetic field strength where a first magnetization of the magnetic particles is not saturated and a second sub-zone has a higher magnetic field strength where a second magnetization of the magnetic particles is saturated,
a drive apparatus comprising:
a drive field signal generator unit; and
a drive coil,
wherein the drive coil is arranged to change the position in space of the first subzone and the second subzone,
wherein the drive coil uses a magnetic drive field to arrange local changes of the first magnetization and the second magnetization of the magnetic particles changes; and
a control unit arranged to control the drive apparatus to change the position in space of the first subzone and the second subzone such that after administration of a drug substance the first sub-zone is moved through a surrounding area disposed around a target area,
wherein the drug substance comprises a drug and magnetic particles.

2. The apparatus as claimed in claim 1, wherein the control unit is arranged to control the drive apparatus to change the position in space of the first subzone and the second subzone such that the first sub zone is moved at least one time or multiple times around the target area.

3. The apparatus as claimed in claim 1, wherein the control unit is arranged to control the drive apparatus to change the position in space of the first subzone and the second subzone such that the first sub zone scans the surrounding area around the target area,
wherein the direction of movement is changed several times.

4. The apparatus as claimed in claim 1, wherein the control unit is arranged to control the drive apparatus and/or the selection apparatus to provide a stationary magnetic field, over the target area and at least a portion of the surrounding area during administration of the drug substance.

5. The apparatus as claimed in claim 1, wherein the control unit is arranged to control the drive apparatus to change the position in space of the first subzone and the second subzone such that the first sub-zone is moved through the surrounding area until the concentration of the drug substance within the surrounding area is below a predetermined threshold.

6. The apparatus as claimed in claim 1, further comprising a receiving apparatus comprising:
a signal receiving unit; and
a receiving coil,
wherein the receiving coil is arranged to acquire detection signals,
wherein the detection signals depend on the first magnetization and the second magnetization in the field of view,
wherein the first magnetization and the second magnetization are influenced by the change in the position in space of the first subzone and the second subzone.

7. The apparatus as claimed in claim 6, wherein the drive apparatus and said receiving means are combined into drive and receiving means comprising a drive-receiving coil, the drive receiving coil is arranged both for changing the position in space of the two sub-zones in the field of view by means of a magnetic drive field and for acquiring detection signals.

8. The apparatus as claimed in claim 6, further comprising a processing unit for reconstructing a spatial distribution and/or a concentration of the magnetic particles within the surrounding area from the detection signals and for determining if the concentration of the drug substance within the surrounding area is below a predetermined threshold.

9. The apparatus as claimed in claim 1, wherein the selection apparatus is arranged to change the stationary gradient of the magnetic selection field and/or the size of the first sub-zone while the first sub-zone is moved through the surrounding area.

10. The apparatus as claimed in claim 1, wherein the control unit is arranged to control the drive apparatus to move the first sub-zone through the surrounding area immediately after the administration of the drug substance.

11. A method for controlling an apparatus for targeted drug delivery the method comprising:
generating a magnetic selection field, wherein the magnetic selection field has a pattern, formed in a field of view, of its magnetic field strength such that a first sub-zone has a low magnetic field strength where a first magnetization of the magnetic particles is not saturated and a second sub-zone has a higher magnetic field strength where a second magnetization of the magnetic particles is saturated,
changing the position in space of the first subzone and the second subzone using a magnetic drive field so that the first magnetization and the second magnetization of the magnetic particles changes,
controlling the changing of the position of the first subzone and the second subzone such that after administration of a drug substance the first sub-zone is moved through a surrounding area disposed around a target area
wherein the drug substance comprises drug substance comprising a drug and magnetic particles.

12. The method as claimed in claim 11,
wherein the drug substance comprises an anticancer agent as drug,
wherein the drug is selected from the group consisting of radionuclides, cancer-specific antibodies, cytostatika and genes.

13. The method as claimed in claim 11, wherein the drug substance comprises multi-domain magnetic particles.

14. The method as claimed in claim 13,
wherein the particles have a maximum dimension through the volume of the particle,
wherein the maximum dimension is at least 100 nm.

15. (canceled)

16. The method as claimed in claim 11,
wherein the drug substance comprises a multi-domain magnetic cluster of particles,
wherein the cluster of particles have a maximum dimension through the volume of the cluster of particles,
wherein the maximum dimension is at least 100 nm.

17. The apparatus as claimed in claim 1, wherein the surrounding area is disposed at a predetermined maximal distance from the target area

18. The method as claimed in claim 11, wherein the surrounding area is disposed at a predetermined maximal distance from the target area

19. The apparatus as claimed in claim 3, wherein the path of the first sub-zone is changed several times.

20. The apparatus as claimed in claim 4, the stationary magnetic field is a stationary magnetic gradient field.
